(19) United States
(12) Patent Application Publication
(10) Pub. No.: US 2006/0172012 A1
(43) Pub. Date: Aug. 3, 2006

(54) ANTI-INFLAMMATORY SUPPLEMENT COMPOSITIONS AND REGIMENS TO REDUCE CARDIOVASCULAR DISEASE RISKS

(76) Inventors: John Westcott Finley, Lansdale, PA (US); Igor Mezine, Malvern, PA (US); John Weldon Finley, Grand Forks, ND (US); Song Gao, Edison, NJ (US); Irene Hoyt, Princeton, NJ (US); Mary M. Opet, Hopewell, NJ (US); Huizhen Zhang, Chalfont, PA (US)

Correspondence Address: THADDIUS J. CARVIS, 102 NORTH KING STREET, LEESBURG, VA, 20176, US

(21) Appl. No.: 11/275,717

(22) Filed: Jan. 25, 2006

Related U.S. Application Data
(60) Provisional application No. 60/647,846, filed on Jan. 28, 2005.

(51) Int. Cl. A61K 35/60; A61K 36/82; A61K 36/45; A61K 36/906; A61K 36/73; A61K 36/87; A61K 31/355

(52) U.S. Cl. 424/523; 424/729; 424/732; 424/766; 424/756; 424/765; 514/458

(57) ABSTRACT

Disclosed are improvements in human nutrition involving a unique combination of natural products constituting anti-inflammatory compositions which can reduce cardiovascular disease risks as well as play a positive role in other conditions and diseases for which key indicators, especially selected from the group consisting of C-reactive protein (CRP) levels, cyclooxygenase-2 (COX-2), 5-lypoxygenase (5-LOX) expression and prostaglandin E2 (PGE-2) biosynthesis or any combination of these, are indicators. Therapeutic compositions preferably comprise curcumin, bilberry extract, grape seed extract, green tea extract and apple extract, in effective amounts individually and combined to provide a therapeutically significant reduction in one or more key indicators. Another exemplified therapeutic composition comprises: omega-3 rich refined fish oil, resveratrol, blueberry extract, grape seed extract, green tea extract and gamma and/or delta tocopherol, in effective amounts individually for the above benefits.

### RELATED APPLICATIONS

This application claims priority to U.S. Patent Application No. 60/647,846, filed Jan. 28, 2005 by John W. Finley and Igor Mezine, the disclosure of which is hereby incorporated by reference in its entirety.

### BACKGROUND OF THE INVENTION

The invention relates to improvements in human nutrition involving providing unique combinations of natural products constituting anti-inflammatory and/or anti-oxidant compositions which can reduce chronic inflammatory conditions such as those related to cardiovascular disease as well as play a positive role in other conditions, especially those that are consequences of central adiposity, and/or related chronic conditions.

Chronic inflammation is strongly related to many diseases and conditions associated with aging. It is also associated with many conditions including arthritis, some forms of cancer, gastric reflux disease, colitis, Alzheimer's disease, immune dysfunction and/or cardiovascular disease. There is a great need for effective treatments, to prevent or reduce inflammatory conditions especially treatments that will be simple and effective and will have little or no adverse side effects.

Chronic inflammation can result from many conditions including central adiposity, periodontal disease, diabetes, chronic infections, stress, and/or environmental influences. Among the major environmental health factors related to chronic inflammation are diet and/or poor nutrition. In particular, diets high in glycemic foods including simple sugars and starches, and diets high in saturated trans fat are related to chronic inflammation.

Chemical oxidation occurs in food products, but it also occurs in vivo. Chemical oxidation of polyunsaturated lipids in vivo can cause the formation of reactive oxygen species and the production of nitric oxide. These end products are associated with early stages of inflammation and can lead to chronic inflammation. Additionally, these reactive oxygen species can result in DNA damage. These products are measured by free radical methods including electron spin resonance, electron paramagnetic resonance, direct measurement of malondialdehyde or the TBARs assay.

Two major enzymatic pathways, the Lipoxygenase pathway and the cyclooxygenase pathway, result in oxidation and inflammatory responses in vivo. The lipoxygenase pathway, particularly the 5-lipoxygenase pathway (5-LOX), results in the production of leukotrienes. Leukotrienes are proinflammatory and are particularly potent in altering immune responses. The cyclooxygenase pathway includes two major factors Cylooxygenase-1 (COX-1) and cyclooxygenase-2 (COX-2). The COX-1 isozyme is primarily a housekeeping enzyme and exists in many healthy cells. The COX-2 enzyme, when induced associated with inflammation. The major products of COX-2 activity are prostaglandins, particularly prostaglandin E2 (PGE-2). COX-2 is important as it relates to infection and other normal inflammatory responses in the body. However, consistently high levels of COX-2 associated with chronic inflammation can lead to a cascade of events that can result in chronic diseases. Down regulation and/or partial inhibition of 5-LOX and COX-2 can help control the negative effects of chronic inflammation.

A frequent symptom of chronic inflammation is high levels of C-reactive protein (CRP). CRP is one of a number of plasma proteins known as acute-phase proteins, i.e., proteins whose plasma concentrations increase (or decrease) by 25% or more during inflammatory disorders. Because CRP levels can rise as high as 1000-fold with inflammation, it is considered a good marker for the presence of inflammation. Other key indicators of inflammation include expression of COX-2, 5-LOX, and, tumor necrosis factor alpha (TNF-α), nuclear factor κB (NF-κB), interluken-6 (IL-6) and interluken-1-β (IL1-β). These enzymes, cytokines and their metabolic products result in the increased production of leukotrien and prostaglandin markers. These leukotriens and prostaglandins are regulatory compounds that are released by cells and act as intercellular mediators or signals in the generation of responses that have been related to inflammation in mammals, particularly humans, and can be measured as serum components.

Recent research has shown the need for treatments for COX-2 mediated inflammation or inflammation-associated disorders. The prostaglandins are a class of biologically active lipid derivatives that have been identified as playing a role in mammalian inflammatory response. The inflammatory response is a localized tissue response to injury or other trauma characterized by pain, heat, redness and swelling. Prostaglandins are implicated in mediating this response by inhibiting platelet aggregation, increasing vascular permeability, increasing vascular dilation, inducing smooth-muscle contraction and/or causing the induction of neutrophil chemotaxis. Prostaglandins are a group of oxygenated fatty acid products that are generally derived from arachidonic acid. The biosynthesis of prostaglandins from arachidonic acid can occur by pathways that can include COX-2 activity resulting in PGE-2 biosynthesis.

Two gene products possessing COX enzyme expression activity are the COX-1 and COX-2 enzymes. COX-1 was the first discovered isoform and is constitutively expressed in most tissue types. COX-1 is available to participate in activities requiring a rapid physiological response by stimulating the production of prostaglandins involved in “house-keeping” functions. COX-2, discovered later, is inducibly expressed in response to numerous stimuli such as bacterial lipopolysaccharides, growth factors, cytokines, and phorbol esters. In addition, COX-2 is only expressed in a limited number of cell types including monocytes, macrophages, neutrophils, fibroblasts and endothelial cells.

COX-2 expression, but not COX-1 expression, has been shown to increase in rheumatoid synovial tissue. Contrastingly, COX-2 expression is inhibited in response to glucocorticoids and by anti-inflammatory cytokines. Thus, based upon these observations, COX-2 has been shown to be the isoform responsible for mediating the production of prostaglandins that participate in the inflammatory response and in inflammatory related disorders. In addition, COX-2 has also been shown to participate in certain cancers, Alzheimer's disease, atherosclerosis, and central nervous system damage resulting from stroke, ischemia and/or trauma.

Adipose tissue, particularly adipose tissue associated with central adiposity, produces increased levels of the inflammatory cytokines TNF-α and IL-6. These inflammatory cytokines then become systemic triggers to inflammation. Elevated levels of these cytokines are frequently associated with increased levels of CRP, mentioned earlier.

CRP is strongly associated with increased risk of cardiovascular disease (CVD). Inflammation is the body's response to injury. Laboratory evidence and clinical and population studies suggest that inflammation is important in atherosclerosis. CRP, as noted, is one of the acute phase proteins that increases markedly during systemic inflammation, making it a good marker.

It has been reported that testing CRP levels in the blood is possibly a way to assess cardiovascular disease risk. A high sensitivity assay for CRP test (hs-CRP) has been related to recurrent incidences of cardiovascular disease, stroke and death in different settings. High levels of hs-CRP can predict new coronary events in patients with unstable angina and acute myocardial infarction (heart attack). Higher hs-CRP levels have also been associated with lower survival rates for these people. Many studies suggest that after adjusting for other prognostic factors, hs-CRP is still useful as a risk predictor. Recent studies also suggest that higher levels of hs-CRP may be associated with increased risk that an artery will reclose after opening by balloon angioplasty. High levels of hs-CRP in the blood seem to predict prognosis and recurrent events in patients with stroke and peripheral arterial disease. Most studies show that the higher the hs-CRP levels, the higher the risk of developing heart disease. In fact, scientific studies have found that the risk for heart attack in people in the upper third percentile of hs-CRP levels is twice that of those whose hs-CRP is in the lower third. Some studies have also found an association between sudden cardiac death, and/or peripheral arterial disease and high levels of hs-CRP. See, for example, Paul M Ridker, M D, MPH, Rosuvastatin in the Primary Prevention of Cardiovascular Disease Among Patients With Low Levels of Low-Density Lipoprotein Cholesterol and Elevated High-Sensitivity C-Reactive Protein, Circulation. 2003;108:2292.

A study at Cleveland Clinic shows that intensive use of statins, or cholesterol-lowering medications, reduces the progression of plaque buildup in the coronary arteries and is highly associated with reduction of CRP, a measure of inflammation in the arteries. A related analysis at Boston's Brigham and Women's Hospital demonstrates that lowering CRP levels with statins also reduces the risk of recurrent heart attack. Both studies appear in the Jan. 6, 2005 issue of the New England Journal of Medicine (NEJM). Like all medications, statins have potential side effects. Although statins are well tolerated by most people, the most common side effects are: nausea, diarrhea, constipation, and/or muscle aching. In addition, two potentially serious side effects are elevated liver enzymes and statin myopathy. Occasionally, statin use causes an increase in liver enzymes aspartate aminotransferase (AST), alanine aminotransferase (ALT). Statins may cause muscle pain and tenderness (statin myopathy). In severe cases, muscle cells can break down (rhabdomyolysis) and release a protein called myoglobin into the bloodstream. Myoglobin can impair kidney function and lead to kidney failure. The U.S. FDA has recently declined to permit over the counter sales of statins for lowering cholesterol.

The gastrointestinal tract is vulnerable to inflammatory responses throughout its entire length. It is highly desirable to prevent or reduce the intensity of inflammation in these tissues. When not controlled, these conditions can become debilitating as they advance from minor irritation, to chronic inflammation, to disease states ranging from acid reflux disease, to colitis, to irritable bowel syndrome, to polyposis. Ultimately these conditions can advance to cancers if not controlled. Typically acid reflux is treated by controlling the proton pump mechanism with drugs such as Nexium. Unfortunately some patients have adverse side effects from such treatment. It would be advantageous to significantly reduce the inflammatory symptoms with minimal side effects.

A wide variety of foods and food extracts, some of which are known to possess antioxidant activity, have been identified as affecting serum levels of various prostaglandins and cytokines; however, the art does not provide a specific direction to one skilled in the art to directly, effectively treat inflammation-related conditions by the use of a satisfactory combination or combinations of these foods and food extracts.

The literature is replete with references to various food materials having some effect on CRP levels and/or cytokine markers; however, there is currently no approved therapy for general use without a prescription which is safe and effective for lowering CRP serum levels and, thereby, having a positive effect on the noted diseases and/or chronic conditions which are associated with elevated CRP serum levels.

### BRIEF DESCRIPTION OF THE INVENTION

It is an object of the invention to provide compositions and a therapy for lowering chronic inflammation, particularly responses that increase CRP serum levels and, thereby, having a positive effect on the noted diseases and/or chronic conditions.

It is an object of the invention to provide compositions and a therapy that reduces the expression and/or activity of enzymes in the COX-2 pathway.

It is an object of the invention to provide compositions and a therapy that reduces the expression and/or activity of enzymes in the 5-LOX pathway, thereby reducing the presence of prostaglandin end products as measured in the serum and urine.

It is an object of the invention to provide compositions and a therapy that reduces the production of IL-6.

It is an object of the invention to provide compositions and a therapy that reduces the production of IL-1β.

It is an object of the invention to provide compositions and a therapy that reduces the expression of interleukins TNF-α and IFN-γ and related inflammatory pathways.

It is an object of the invention to provide compositions and a therapy that reduces the expression of NFκB and related inflammatory pathways.

It is an object of the invention to provide compositions and a therapy that reduces the expression of iNOS in endothelial tissue and related inflammatory pathways.

It is an object of the invention to provide compositions and a therapy that reduces the expression of COX-2 and/or PGE-2 production in cardiac smooth muscle tissue.

It is an object of the invention to provide compositions and a therapy that reduces oxidative markers in the plasma or serum as measured by TBARS.

It is an object of the invention to provide compositions and a therapy that reduces the expression of selectins ICAM-1 and VCAM-1.

It is an object of the invention to provide specific combinations of ingredients or food extracts that deliver combinations of bioactive compounds which directly, effectively treat inflammation-related conditions.

It is another object of one aspect of the invention to provide improvements in human nutrition by identifying new combinations of natural products useful in treating side effects caused by fat deposits in mammals and, specifically, treating those affecting central adiposity.

It is another object of another aspect of the invention to provide improvements in human nutrition by identifying new combinations of natural products useful in reducing serum oxidative stress indicators which in turn reduce CVD risks.

It is yet another specific object of one aspect of the invention to provide compositions and therapies to treat acid reflux by natural food materials in dosage form as opposed to controlling the proton pump mechanism with drugs that can cause side effects in some patients.

It is yet another object of the invention to provide a variety of product forms for new combinations of compositions, in particular specially formulated foods containing natural products useful in treating the conditions identified above.

These and other objects are achieved by the invention, which provides therapeutic compositions comprising unique combinations of natural products constituting anti-inflammatory supplement compositions and regimens employing them to reduce cardiovascular disease risks and/or other conditions and diseases such as chronic inflammation which elevate inflammatory markers including CRP.

The invention provides therapeutic compositions and regimens comprising unique combinations of natural products to provide a therapy for decreasing causes of inflammation, especially as evidenced by lowering CRP serum levels and, thereby, having a positive effect on diseases and/or related chronic conditions which result in elevated CRP serum levels, the compositions and regimens based on specific combinations of food extracts. These compositions can be considered as improvements in human nutrition in that they present a new combination of natural products useful in reducing CVD risks.

The therapeutic compositions provided by the invention comprise a unique combination of natural products constituting anti-inflammatory supplement compositions, the regimens employing them are effective to reduce CVD risks and/or other conditions and diseases such as chronic inflammation which elevate markers including CRP.

In a preferred form the invention provides a therapeutic composition according to claim 1, wherein the food extracts are selected from the group of apple extract, green tea extract, curcumin, bilberry extract, blueberry extract, mixed tocopherols, resveratrol, omega-3 rich oils and grape seed extract, containing compositions with anti-inflammatory activity, the compositions being present in amounts individually and combined to provide a therapeutically significant reduction of at least two of the markers selected from the group of CRP, COX-2, 5-LOX, TNF-α, NF-κB, IL-6 and IL1-β. As will be exemplified below among other formulations, one preferred composition of the invention comprises curcumin, bilberry extract, grape seed extract, green tea extract and apple extract.

In another exemplified form, the composition of the invention can comprise omega-3 rich refined fish oil, resveratrol, blueberry extract, grape seed extract, green tea extract, gamma and delta tocopherol mixture.

In another form, composition of the invention can comprise a mixture of green tea extract, e.g., from 30 to 60%, grape seed extract, e.g. from 20 to 40%, apple extract, e.g., from 10 to 20% and curcumin, e.g., from 2 to 10% as a dry powder. This form of mixture can be introduce into a variety of foods. One formulation exemplified below comprises a mixture of 48.7% green tea extract, 30.7% grape seed extract, 14.6% apple extract, and 6.0% curcumin. Another exemplified composition of the invention comprises a mixture of 38.0% green tea extract, 24.0% grape seed extract, 21.9% bilberry extract, 11.4% apple extract, and 4.7% curcumin.

Typically, the compositions of the invention include active ingredients from among the curcuminoids, proathocyanidins, quercetin and the catechins. In addition, because these compounds are phenolics they can act as antioxidants. In this function along with the tocopherols they can break oxidative chain reactions and quench reactive oxygen species and nitric oxide. This antioxidant function removes these radicals from the system thus lowering one source of initiators of inflammation. The invention is, however, more specific than simple antioxidant therapy and can be effective in reducing markers of chronic inflammation. Indeed, in preferred forms, the compositions of the invention reduce the expression and/or activity of enzymes in the COX-2 pathway, thereby reducing the presence of prostaglandin end products as measured in the serum and urine. They can also reduce 5-LOX activity in vivo and reduce the expression and/or activity of enzymes in the 5-LOX pathway thereby reducing the presence of prostaglandin end products as measured in the serum and urine.

In other preferred forms of this invention, the combination of ingredients including mixed tocopherols (α, β, γ and/or δ) results in the reduction of COX-2 from multiple causes as well as interference of COX-2 enzyme activity. Because these materials are also antioxidants their scavenging of reactive oxygen species in the inflamed tissue can also slow progression of the arthritic condition.

In a preferred form, the invention provides a therapeutic composition comprising extracts of natural materials identified above, e.g., apple extract and green tea extract, especially with curcumin and preferably also with bilberry extract and/or grape seed extract, containing key compositions or classes of compositions (from the list in Table 1, below) present in amounts individually and combined to provide a therapeutically significant reduction of one or more of the following as markers of inflammation: CRP, COX-2, 5-LOX, TNF-α, NF-κB, IL-6 and IL1-β.

In another specific aspect of the invention, there is provided a mixture comprised of curcumin, green tea extract, grape seed extract, γ-tocopherols and blueberry and/or bilberry extract in amounts effective to reduce inflammation in the upper GI tract. The effectiveness can be enhanced by the addition of a modified lipid composed of triglycerides appended with short chain fatty acids and preferably long chain fatty acids containing omega-3 fatty acids and/or monounsaturated fatty acids are included in the treatment. These lipids are quickly digested, the butyric acid is used as energy by the epithelial cells and the omega-3 fatty acids act as anti-inflammatory agents in conjunction with the botanical materials.

Preferred aspects of the invention will be described and illustrated below.

### DETAILED DESCRIPTION OF THE INVENTION

The invention provides therapeutic compositions and regimens comprising unique combinations of natural products. It is an object of the invention to provide a therapy for decreasing causes of inflammation, especially as evidenced by lowering CRP serum levels and, thereby, having a positive effect on diseases and/or related chronic conditions which result in elevated CRP serum levels. The invention can effectively treat inflammation-related conditions by the use of a specific combination or combinations of food extracts. These compositions can be considered as improvements in human nutrition in that they present new combinations of natural products useful in reducing CVD risks.

The therapeutic compositions provided by the invention comprise unique combinations of natural products constituting anti-inflammatory supplement compositions, the regimens employing them are effective to reduce CVD risks and/or other conditions and diseases such as chronic inflammation which elevate markers including CRP.

In a preferred form the invention provides a therapeutic composition according to claim 1, wherein the food extracts are selected from the group of apple extract, green tea extract, curcumin, bilberry extract, blueberry extract, mixed tocopherols, resveratrol, omega-3 rich oils and grape seed extract, containing compositions with anti-inflammatory activity, the compositions being present in amounts individually and combined to provide a therapeutically significant reduction of at least two of the markers selected from the group of CRP, COX-2, 5-LOX, TNF-α, NF-κB, IL-6 and IL1-β. As will be exemplified below among other formulations, one composition of the invention comprises 50 parts curcumin, 60 parts bilberry extract, 250 parts grape seed extract, 375 parts green tea extract, 125 parts apple extract. The doses and these individual ingredients can be varied by up to 50% of the above values, preferably varying by no more than 25%. The purity of the ingredients and the presence of added diluents, emulsifiers and other additives must be taken into consideration in determining the dosage.

In another exemplified form, the composition of the invention can comprise 500 mg omega-3 rich refined fish oil, 100 mg resveratrol, 150 mg blueberry extract, 100 mg grape seed extract, 50 mg green tea extract, 100 mg gamma and delta tocopherol mixture (deodorize distillate). A dosage unit of this formulation will preferably be from 0.5 to 2.0 grams. And, again, the doses and these individual ingredients can be varied by up to 50% of the above values, preferably varying by no more than 25%.

In anther form, composition of the invention can comprise a mixture of green tea extract, e.g., from about 30 to about 60%, grape seed extract, e.g. from about 20 to about 40%, apple extract, e.g., from about 10 to about 20% and curcumin, e.g., from about 2 to about 10% as a dry powder. This form of mixture can be mixed in a prepared meal, dip, or soup, or added before cooking/microwaving the meal or soup and is well delivered in Chili, taco and southern or southwestern style meals and soups. One formulation exemplified below comprises a mixture of 48.7% green tea extract, 30.7% grape seed extract, 14.6% apple extract, and 6.0% curcumin. Another exemplified composition of the invention comprises a mixture of 38.0% green tea extract, 24.0% grape seed extract, 21.9% bilberry extract, 11.4% apple extract, and 4.7% curcumin.

The combinations of ingredients that are effective according to preferred forms of this invention can reduce CRP and enzyme activities in the 5-LOX and COX-2 pathways and preferably provide a therapeutically significant reduction of at least two of the markers selected from the group of COX-2, 5-LOX, TNF-α, NF-κB, IL-6 and IL1-β.

Typically, the compositions of the invention include active ingredients from among the curcuminoids, proathocyanidins, quercetin and the catechins. In addition, because these compounds are phenolics they can act as antioxidants. In this function along with the tocopherols they can break oxidative chain reactions and quench reactive oxygen species and nitric oxide. This antioxidant function removes these radicals from the system thus lowering one source of initiators of inflammation. The invention is, however, more specific than simple antioxidant therapy and can be effective in reducing markers of chronic inflammation. Indeed, in preferred forms, the compositions of the invention reduces the expression and/or activity of enzymes in the COX-2 pathway, thereby reducing the presence of prostaglandin end products as measured in the serum and urine. They can also reduce 5-LOX activity in vivo and reduce the expression and/or activity of enzymes in the 5-LOX pathway thereby reducing the presence of prostaglandin end products as measured in the serum and urine.

The activities of the compositions of the invention have significant consequence for diseases and conditions related to chronic inflammation. For example, joint pain from rheumatoid and osteoarthritis has inflammatory components and can be treated by the compositions and regimens of the invention. The reduction of COX-2 expression or the reduction of COX-2 activity reduces the joint inflammation and resulting pain. The mixtures that are the object of this invention reduce both the expression of COX-2 as well as reduce the activity of the expressed enzyme. Reduction of expression of COX-2 can be the indirect consequence of reduced expression of TNF-α or NF-κB.

In other preferred forms of this invention, the combinations of ingredients including mixed tocopherols result in the reduction of COX-2 from multiple causes as well as the interference of the enzyme activity and suppression of COX-2 gene suppression. Because these materials are also antioxidants the scavenging of reactive oxygen species in the inflamed tissue can also slow progression of the arthritic condition.

As noted above, the gastrointestinal tract is vulnerable to inflammatory responses throughout its entire length, and it is an advantage of the invention that the compositions and regimens provided can be useful in preventing or reducing the intensity of inflammation in these tissues. When not controlled, these conditions can become debilitating as they advance from minor irritation, to chronic inflammation, to disease states ranging from acid reflux disease, to colitis, to irritable bowel syndrome, to polyposis. Ultimately these conditions can advance to cancers if not controlled.

It is an advantage that acid reflux is treated by the invention as opposed to controlling the proton pump mechanism with drugs such as Nexium, which can cause adverse side effects in some patients. Through the use of mixed mild anti-inflammatory materials according to the invention it is possible to significantly reduce the symptoms with minimal side effects. A mixture comprised of curcumin, green tea extract, grape seed extract, γ-tocopherol and blueberry and/or bilberry extract can be effective in reducing inflammation in the upper GI tract. The effectiveness can be enhanced if a modified lipid composed of triglycerides appended with short chain fatty acids and preferably long chain fatty acids containing omega-3 fatty acids are included in the treatment. These lipids are quickly digested, the butyric acid is used as energy by the epithelial cells and the omega-3 fatty acids act as anti-inflammatory agents in conjunction with the botanical materials. In this regard, reference is made to U.S. patent application Ser. No. 11/275,495, the disclosure of which is incorporated by reference in its entirety.

Relief of inflammation in the small intestine can be achieved by delivering the anti-inflammatory mixture in a matrix such as a protein or starch complex that is poorly digested in the stomach but efficiently digested in the intestine. A similar anti-inflammatory mixture as described above can be used for this purpose if it is included in a protein matrix. Such materials can be achieved by microencapsulation. For this purpose, resveratrol or pycnogenol can be included in the complex. For treatment or prevention of inflammation in the lower GI tract and colon cancer the poor absorption of curcumin, resveratrol and grape seed extract are effective in a variety of forms, especially delivering them in conjunction with an insoluble fiber such as an insoluble pectin or cellulose. The colonic bacteria will release the bioactives and they will be absorbed by the colon cells and there they will prevent or inhibit the progression of inflammatory conditions.

In a preferred form, the invention provides therapeutic compositions comprising extracts of natural materials identified above, e.g., apple extract and green tea extract, especially with curcumin and preferably also with bilberry extract and/or grape seed extract, containing key compositions or classes of compositions (from the list in Table 1, below) present in amounts individually and combined to provide a therapeutically significant reduction of one or more of the following as markers of inflammation: CRP, COX-2, 5-LOX, TNF-α, NF-κB, IL-6 and IL1-β.

The natural extracts deliver at least six, and preferably more, of the bioactive compositions listed in Table 1.

TABLE 1

1,8-Cineole
4-terpineol
Allantoin
α-Amyrin
Anthocyanidins:
Delphinidin
Cyanidin
Petunidin
Peonidin
Malvidin
Pelargonidin
Anthocyanins
Apigenin
Apigenin-7-o-rutinoside
ar-Turmerone
Ascorbic acid
α-Terpineol
Avicularin
Benzoic acid
β-Carotene
β-Ionone
Borneol
β-Pinene
β-Sitosterol
Caffeic acid
Caffeine
Caffeoylquinic acid
Carvacrol
Caryophyllene
Catechins:
(+)-Catechin
(−)-Epicatechin (EC)
(+)-Gallocatechin (GC)
(−)-Epigallocatechin (EGC)
(−)-Epicatechin gallate (ECG)
(−)-Gallocatechin gallate
(GCG)
(−)-Epigallocatechin gallate
(EGCG)
Chlorogenic acid
Cinnamic acid
Citric acid
Curcuminoids:
Curcumin
Demethoxycurcumin
Bis-demethoxycurcumin
Docosahexaenoic acid (DHA)
Eicosapentaenoic acid (EPA)
Ellagic acid
Ellagic Acid glycosides
Eriodictyol-7-o-rutinoside
Eugenol
Farnesol
Ferulic acid
Fumaric acid
Gallic acid
Gentistic acid
Geraniol
Guaiacol
Hyperin
Hyperoside
Isoquercitrin
Kaempferol
Libiatae extracts
Linolenic acid
Lupeol
Lutein
Luteolin
Luteolin-diglucuronide
Luteolin-7-o-glucuronide
Luteolin glycoside
Luteolin-7-o-rutinoside
Lycopene
Monotropein
Myricetin
Naringenin
Neo-chlorogenic acid
o-Coumaric acid
p-Coumaric acid
p-coumaroyl quinic acid
Phloretin
Phloretin-xyloglucoside
Phloridzin
p-Hydroxy benzoic acid
Proanthocyanidins
Protocatechuic acid
Punicalins
Punicalagin A
Punicalagin B
Pycnogenol
Quercetin
Quercitrin
Resveratrol
Rosmarinic Acid
Rutin
Salicin
Salicylic acid
Selenium
Syringic acid
Terpineol
Theaflavins:
Theaflavin
Theaflavin-3-gallate
Theaflavin-3′-gallate
Theaflavin-3,3′-digallate
Theobromine
Theophylline
Thiamin
Thymol
Tocopherols:
α-Tocopherol
β-Tocopherol
γ-Tocopherol
δ-Tocopherol
Ursolic acid
Vanillic acid
Vitexin
Vitexin-2-rahmnoside

Tables 2 through 6 present listings of preferred, exemplary food extracts and the key compositions or classes of compositions contributed by them.

TABLE 2
Apple Extract

α-linolenic acid
α-tocopherol
Ascorbic acid
Avicularin
β-carotene
Caffeic acid
(+)-Catechin
Chlorogenic acid
Citric acid
(−)-Epicatechin
Ferrulic acid
Fumaric acid
Geraniol
Hyperin
Hyperoside
Isoquercitrin
Lutein
p-coumaric acid
p-coumaroyl quinic
acid
p-hydroxy-benzoic acid
Phloretin
Phloretin-xyloglucoside
Phloridzin
Proanthocyanidins
Procyanidin B1
Procyanidin B2
Procyanidin C1
Quercetin
Quercitrin
Protocatechic acid
Rutin
Ursolic acid

TABLE 3
Grape Seed Extract

Anthocyanidine
Anthocyanins
Benzoic acid
(+)-catechin
Caffeic acid
(−)-epicatechin
flavan-3-ol
Gallic acid
gentistic acid
Proanthocyanidins
Procyanidin B1
Procyanidin B2
Procyanidin B3
Procyanidin B4
Procyanidin C1
Protocatechulic acid
Vanillic acid
Syringic acid

TABLE 4
Turmeric Extract

α-terpineol
AR-turmerone
Ascorbic acid
β-carotene
Bis-demethoxycurcumin
β-pinene
Borneol
Caffeic acid
Caryophyllene
1,8-Cineole
Cinnamic acid
Curcumin
Demethoxycurcumin
Eugenol
Guaiacol
p-coumaric acid
protocatechuic acid
Syringic acid
Terpineol

TABLE 5
Bilberry Extract

Anthocyanins
Ascorbic acid
Benzoic acid
β-carotene
Caffeic acid
Chlorogenic accid
Cyanidin
Delphinidin
Ferulic acid
Hyperoside
Lutein
Malvidin
Monotropein
o-coumaric acid
p-Hydroxy-benzoic acid
Peonidin
Pelargonidin
Petunidin
Protocatechuic
acid
Selenium
Syringic acid
Thiamin
Ursolic acid
Vanillic acid

TABLE 6
Tea Extract

4-terpineol
α-amyrin
Allantoin
α-amyrin
α-Terpineol
Benzoic acid
β-Ionone
β-Sitosterol
Caffeic acid
Caffeine
Carvacrol
(+)-catechin
Chlorogenic acid
Cinnamic Acid
(−)-epicatechin
(−)-epicatechin gallate
(−)-epigallocatechin
(−)-epigallocatechin gallate
Eugenol
Farnesol
Gallic acid
(+)-gallocatechin
(−)-epigallocatechin gallate
Geraniol
Hyperoside
Kaempferol
Lycopene
Lupeol
Lutein
Myricetin
Naringenin
Neo-chlorogenic acid
Quercetin
Rutin
Salicylic acid
Theaflavin
Theaflavin-3-gallate
Theaflavin-3′-gallate
Theaflavin-3,3′-digallate
Theobromine
Theophyline
Thiamin
Thymol
Vitexin

The compositions of the present invention can include an effective amounts of a combination of natural extracts, such as bilberry extract containing anthocyanins, green tea extract containing catechins, grape seed extract containing proanthocyanidins and catechins, apple extract containing catechins, proanthocyanidins, chlorogenic acid, phloretin, phloridzin, and flavinoids, and turmeric extract containing curcuminoids. The various food extracts identified as useful herein are available commercially or have been identified in the patent and other literature in various forms with various activities. The extracts can be made using techniques involving solvent extraction and concentration to yield extracts having concentrations of active materials, particularly at least one of those listed in Table 7 below for the particular extracts of at least 50 times, typically at least 100 times, and preferably at least 250 times, those present in the starting food materials. It will be seen that the extracts can be used in the formulations of the invention in amounts of from about 10% to about 500% of the amount present in a typical serving of the starting material (e.g., about 100 gram serving size in the case of fruits and a six ounce cup of 3 minute 180° F. brew from one teaspoon of tea for green tea). In some cases, such as apple extract, the amount is closer to an about 50% level and for bilberry and blueberry the amount will be closer to an about 25% level, but both can be higher. All extracts can be made using 0 to 100% water and/or organic solvent (e.g., hexane and/or ethanol) extraction at suitable pH and temperature (e.g., pH can run the range for 1 to 14 and the extraction temperature can be between 0 to 100° C.) preferably followed by optional purification by means of liquid-liquid extraction, solid-phase extract, supercritical carbon dioxide extraction, chromatographic methods, membrane ultrafiltration or combinations of thereof. Water extracts of active compounds derived from green tea are effective. See for example U.S. Pat. No. 6,387,416 for super critical carbon dioxide extracts and U.S. Pat. No. 4,935,256 to Tsai for green tea extracts. In all cases, the extracts can be treated to remove or stabilize color and/or flavor. Also, the Labiatae extracts in the above list can be prepared by aqueous extraction and isolation as described in U.S. Patent Application No. 60/644,738, filed in the names of Igor Mezine, et al.

Exemplary of a suitable range of active compounds in the some of the above extracts useful according to the invention are those set out in Table 7:

TABLE 7

| Food Extract | Active compositions | Concentration |
|---|---|---|
| Green Tea Extract | Catechins | ≧70% (75.9%) |
| | EGCG | ≧50% (53.2%) Total |
| | Caffeine | 3.8% |
| Grape Seed | Catechins | 10.9% |
| Extract | Proanthocyanidins | 90% |
| Apple Extract | Proanthocyanidins | 70% |
| | Quercetin | Up to 1% |
| | Chlorogenic Acid | 12.3% |
| | Phloridzin | 13.9% |
| Bilberry/also | Proanthocyanidins | 50% |
| Blueberry Extract | Anthocyanins | 37% |
| | Delphinidin: | 5.1% |
| | Cyanidin | 4.1% |
| | Petunidin | 4.3% |
| | Peonidin | 0.4% |
| Curcumin | Bis-demethoxycurcumin | 3.5% |
| | Curcumin | 70.3% |
| | Demethoxycurcumin | 18.1% |

As used herein, the term “green tea” refers to leaves obtained from the genus Camellia including C. sinensis and C. assaimica, or their hybrids, for instance, freshly gathered green tea leaves, fresh green tea leaves that are dried immediately after gathering, fresh green tea leaves that have been heat treated before drying to inactivate any enzymes present, unfermented tea, instant green tea, and aqueous extracts of these leaves. According to a preferred form of the invention, green tea extracts are employed at from about 0.25 to about 1 gram per day, e.g., an amount equal to about 3 cups of green tea, but this can be varied by up to 50% and still be highly effective. EGCG @50%=230 mg Green tea materials can include tea leaves, their extracts, tea plant stems and other plant materials which are related and which have not undergone partial or substantial fermentation to create oolong teas. Extracts from white tea, or tea from Camelia sinensis, which has been harvested before the leaves are fully open and subject to little processing and almost no fermentation can also be used. Other members of the genus Phyllanthus, Catechu gambir or Uncaria family of tea plants can also be used. Mixtures of unfermented teas can be also used in preparing green tea extracts useful in the beverages. Alternative sources of the active catechin compositions available from green tea are extracts of oolong tea, white tea, grapes, apple, cocoa beans, and pears, which should be used in amounts sufficient to give equivalent activity to that of the green tea extracts when used in replacement.

Alternative sources of the active compositions for those of bilberry are contained in the extracts of blueberry, cranberry, raspberry, cherry, mulberry, pomegranate, purple corn, strawberry, grapes, black berry, gooseberry and black currants.

Alternative sources of the active compositions for those from grape seed are contained in extracts of cocoa beans, coffee beans, pine bark, cinnamon bark, cranberries, grape skins, lemon tree bark, and hazel nut tree leaves and apple.

The amounts of materials from the lists in Tables 1 through 7 to be used in the compositions of the invention can be determined or monitored by preparing a combination of food extracts comprising two or more of these compositions or groups of compositions and determining effective amounts individually and combined for the composition to provide a therapeutically significant reduction of the following key markers including COX-2, 5-LOX, TNF-α, NF-κB, IL-6 and IL1-β. Testing of serum levels following consumption in a regimen of twice daily doses for one week is effective. Preferably, however, laboratory testing can be conducted in vitro by methods reported in cell model systems. The following discuss useful test methodology:
Meng, Charles Q.; Somers, Patricia K.; Hoong, Lee K.; Zheng, X. Sharon; Ye, Zhihong; Worsencroft, Kimberly J.; Simpson, Jacob E.; Hotema, Martha R.; Weingarten, M. David; MacDonald, Mathew L.; Hill, Russell R.; Marino, Elaine M.; Suen, Ki-Ling; Luchoomun, Jayraz; Kunsch, Charles; Landers, Laura K.; Stefanopoulos, Dimitria; Howard, Randy B.; Sundell, Cynthia L.; Saxena, Uday; Wasserman, Martin A.; Sikorski, James A. AtheroGenics, Inc., Alpharetta, Ga., USA. “Discovery of novel phenolic antioxidants as inhibitors of vascular cell adhesion molecule-1 expression for use in chronic inflammatory diseases”. Journal of Medicinal Chemistry 2004, 47(25), 6420-6432.
Daxs, Claudia I.; Lottspeich, Friedrich; Muellner, Stefan. Biozentrum Niederursel, Johann Wolfgang Goethe Univ., Frankfurt/Main, Germany. “In vitro model system for the identification and characterization of proteins involved in inflammatory processes. Electrophoresis” 1998, 19(10), 1841-1847.
“Ajoene, a natural product with non-steroidal anti-inflammatory drug (NSAID)-like properties.” Dirsch V M; Vollmar A M Institute of Pharmacy, Center of Drug Research, Butenandtstrasse 5-13 B, University of Munich, 81377, Munich, Germany. Verena.Dirsch@cup.uni-muenchen.de Biochemical Pharmacology 2001, 61(5), 587-93.
Shen, S. C.; Lee, W. R.; Lin, H. Y.; Huang, H. C.; Ko, C. H.; Yang, L. L.; Chen, Y. C. “In vitro and in vivo inhibitory activities of rutin, wogonin, and quercetin on lipopolysaccharide-induced nitric oxide and prostaglandin E₂ production.” EUr. J. Pharmacol 2002, 446, 187-194.
Chang, B. W.; Kim, D. H.; Kowalski, D. P.; Burleson, J. A.; Son, Y. H.; Wilson, L. D.; Haffy, B. G. “Prognostic significance of cyclooxygenase-2 in oropharyngeal squamous cell carcinoma.” Clin. Cancer Res. 2004, 10, 1678-1684.
Chan, F. K.; Hung, L. C.; Suen, B. Y.; Wu, J. C.; Lee, K. C.; Leung, V. K.; Hui, A. J.; To, K. F.; Leung, W. K.; Wong, V. W.; Chung, S. C.; Sung, J. J. “Celecoxib versus diclofenac and omeprazole in reducing the risk of recurrent ulcer bleeding in patients with anthritis.” N. Engl. J. Med. 2002, 347, 2104-2110.
Chau, I.; Cunningham, D. “Cyclooxygenase inhibition in cancer: a blind alley or a new therapeutic reality?” New Engl. J. Med. 2002, 346, 1085-1087.
Mutoh, M.; Takahashi, M.; Fukuda, K.; Matsushima-Hibiya, Y.; Mutoh, H.; Sugimura, T.; Wakabayashi, K. “Suppression of cyclooxygenase-2 promoter-dependent transcriptional activity in colon cancer cells by chemopreventive agents with a resorcin-type structure.” Carcinogenesis 2000, 21, 959-963.

In one preferred aspect the invention provides therapeutic compositions comprising: curcumin, bilberry extract, grape seed extract, green tea extract, apple extract, in effective amounts individually and combined to provide a therapeutically significant reduction in serum levels of one or more of the following key markers COX-2, 5-LOX, TNF-α, NF-κB, IL-6 and IL1-β.

One preferred therapeutic composition comprises: 50 parts curcumin, 60 parts bilberry extract, 250 parts grape seed extract, 375 parts green tea extract, 125 parts apple extract. All parts are by weight. A dosage unit of this formulation and others of the invention can preferably be from 0.1 to 5 grams, e.g., 0.5 to 2.0 grams, preferably in gelatin or like capsules for oral administration or as a mixture of ingredients effective for blending into a food. The capsules are desirably consumed in a regimen effective to provide beneficial change in at least one key indicator, e.g., two to four times daily, at regular intervals, preferably taking them once in the morning and once in the evening or at intervals of eight hours. The doses and the individual ingredients can be varied by up to 50% of the above values, preferably varying by no more than 25%. The purity of the ingredients and the presence of added diluents, emulsifiers and other additives must be taken into consideration in determining the dosage

In another aspect, the compositions of the invention will comprise a therapeutic regimen comprising administering a composition of the invention at intervals and in amounts effective to reduce cardiovascular disease risks and/or other conditions and diseases for which elevated CRP and/or other cytokine markers' levels are an indicator. The preferred regimens will be effective to reduce cardiovascular disease risks and/or other conditions and/or related diseases as evidenced by significant change in one of the key indicators. A dosage unit of this formulation will preferably be from 0.5 to 2.0 grams, preferably in gelatin or like capsules for oral administration or can be orally administered as part of a prepared food. The capsules are desirably consumed in a regimen effective to provide beneficial change in at least one key indicator, e.g., two to four times daily, at regular intervals, preferably taking them once in the morning and once in the evening or at intervals of eight hours. The doses and the individual ingredients can be varied by up to 50% of the above values, preferably varying by no more than 25%. The purity of the ingredients and the presence of added diluents, emulsifiers and other additives must be taken into consideration in determining the dosage. In another aspect, the invention, particularly the compounds rich in phenolic components from the list in Table 7, will have activity for treating and, preferably reducing, central adiposity when made a part of a regimen including daily doses as outlined herein.

The above components of the compositions of the invention should have, individually, effective levels of purity to meet the objectives of the invention. The components can be standardized for dosage level based on ORAC assay values (Oxygen Radical Absorbance Capacity assay (commonly referred to as the ORAC assay), with the levels meeting those recommended for daily dosages. The ORAC assay, determines free radical scavenging activity against the peroxyl radical for both water-soluble and lipid-soluble substances. An ORAC-hydro assay reflects water-soluble antioxidant capacity, while an ORAC-lipo assay measures lipid-soluble antioxidant capacity. The values of these two assays are additive. The above ingredients should be employed in total amount to provide a daily intake of at least 3,000 and preferably at least 5,000 ORAC units. Each of the components individually should contribute no less than 500 ORAC units. A therapeutic dosage unit will contain at least 1,000 and preferably at least 1,500 ORAC units, and preferably, each of the components individually contributes no less than 200 ORAC units.

In yet another aspect, the invention will provide a method for determining the effectiveness of an anti-inflammatory therapeutic composition comprised of at least two food extracts containing compositions as listed above in Tables 1 though 6 comprising: formulating a food comprised of at least two food extracts; determining effective in amounts individually and combined for the composition to be effective to provide a therapeutically significant reduction of one or more of the following markers including COX-2, 5-LOX, TNF-α, NF-κB, IL-6 and IL1-β.

To check on the effectiveness of the regimen, the subjects are tested in a suitable clinical evaluation, such as:
If hs-CRP level is lower than 1.0 mg/L, a person has a low risk of developing cardiovascular disease.
If hs-CRP is between 1.0 and 3.0 mg/L, a person has an average risk.
If hs-CRP is higher than 3.0 mg/L, a person is at high risk.

Other key markers of inflammation induced oxidative stress are Advanced Glycation End products (AGE) Clinically relevant AGCE include pentsidine, DeOxygluconsone derived Lysine Dimer (DOLD); Glyoxal derived Lysine Dimer (GOLD); and a number of hydrolmidiazolones denoted as MG-H. Serum pentosidine level in patients with CVD is 28.4 pmol/mg albumin; in healthy group it is 21.4 pmol/mg albumin. The pentosidine level in urine also correlates with DNA damage marker 8-hydroxydeoxyguanosine. The degree of oxidative modifications of proteins can be determined via quantification of corresponding markers such as methionine sulfoxide, otho-tyrosine (o-tyr) and ditryrosine, chlorotyrosine and nitrotyrosine.

Optional therapeutic compositions can contain in addition to the above components, from 1000 mg to 3000 mg of plant-derived sterols or stanol esters. These additional components, also known as phytosterols or phytosterol esters are effective to reduce serum cholesterol in modest amounts. Consumption of a combination of materials including the anti-inflammatory compositions identified in Table 1 results in a formulation that will lower CRP and serum LDL cholesterol, thus lower the risk of CVD. In an expansion of the technology, combining the CRP reducing formulation with a mixture of phytosterols to reduce low density lipids (LDL) cholesterol will further reduce the risk of CVD. Natural antioxidants found in food are preferably combined with the noted anti-inflammatory composition and can further reduce chronic inflammation associated with plaque build-up and CVD.

A combination of ingredients significantly reduce chronic inflammatory conditions. Phytosterol esters, also known as plant sterol (or steryl) esters, are composed of vegetable oil fatty acids and plant lipids (phytosterols) are naturally present in vegetables, fruits, and grains. Phytosterol esters are available for incorporation into oil-based food products as well as other compositions, but the presence of added oil diluents and possibly emulsifiers and other additives must be taken into consideration in determining the dosage. Ingestion of phytosterol esters helps promote healthy blood cholesterol levels. Lipase enzymes, present in the human digestive tract, cleave phytosterol esters to liberate phytosterols in the gastrointestinal tract, which then block gastrointestinal absorption of dietary and biliary cholesterol into the bloodstream, thus lowering serum cholesterol.

The U.S. FDA has approved a claim that daily consumption of at least a total of 1.3 grams of phytosterol esters in two meals may reduce the risk of heart disease when part of a diet low in fat and cholesterol.

In all cases, steps are taken to assure the potency of the components both as formulated and for a prescribed period of time following packaging. In alternative embodiments, one or more of the active components can be optional or reduced in concentration.

Formulations in the form of candies are shown in the Examples and can be made with or without Zinc. The green tea extracts and apple extracts are processed with a knowledge of their sensitivity in high moisture systems. Also there is a need to separate tocopherals and Omega 3 fatty acid containing compositions.

Among other key markers useful in identifying the effectiveness of the compositions of the invention, namely COX-2 and/or PGE-2 biosynthesis, can be determined by assay methods established in the art. See, for example the following for COX-2 methods: Iniguez, M. A.; Punzon, C.; Fresno, M. “Induction of cyclooxygenase-2 on activated T lymphocytes: regulation of T cell activation by cyclooxygenase-2 inhibitors.” J. Immunol. 1999, 163, 111-119; and Romare, A.; Lundholm, C. E. “Cadmium-induced calcium release and prostaglandin E2 production in neonatal mouse calvaria are dependent on cox-2 induction and protein kinase C activation.” Arch. Toxicol. 1999, 73, 223-228. In Example 3 below, we describe one technique which we find particularly effective for PGE-2. Among other literature methods are: Yang P, Felix E, Madden T, Fischer S M, Newman R A., “Quantitative high-performance liquid chromatography/electrospray ionization tandem mass spectrometric analysis of 2- and 3-series prostaglandins in cultured tumor cells.” Anal Biochem. 2002 Sep. 1;308(1):168-77. PMID: 12234478 [PubMed—indexed for MEDLINE]. See also: Newby C S, Mallet A I., “Rapid simultaneous analysis of prostaglandin E2, 12-hydroxyeicosatetraenoic acid and arachidonic acid using high performance liquid chromatography/electrospray ionization mass spectrometry.” Rapid Commun Mass Spectrom. 1997;11(15):1723-7. PMID: 9364799 [PubMed—indexed for MEDLINE].

In variations on the above formulations, the therapeutic compositions can be employed in combination with 0.5 to 5 g of glucosamine to reduce joint inflammation, reduce pain from osteoarthritis and enhance joint health. Additionally, they can contain 0.1 to 1 g of chondroitin sulfate.

In another principal independent embodiment of the invention, there are provided therapeutic compositions comprising: omega-3 rich refined fish oil, resveratrol, blueberry extract, grape seed extract, green tea extract and gamma and delta tocopherol mixture (deodorize distillate), in effective amounts individually and combined to provide a therapeutically significant reduction in CRP and/or other inflammatory markers including cytokines.

Another principal embodiment of the invention provides therapeutic compositions and regimens comprising unique combinations of natural products. The compositions of this embodiment of the invention can comprise omega-3 rich refined fish oil, resveratrol, blueberry extract, grape seed extract, green tea extract and gamma and/or delta tocopherol mixture (deodorize distillate), in effective amounts individually and combined to provide a therapeutically significant reduction in CRP. Also employed as optional, but highly effective, components are stanols and/or stanol esters. Most of the above ingredients can be provided by foods rich in these compositions, and are preferably processed by those means known to the art to preserve and/or enrich the active compounds, it being recognized that the exact compositions having the desired active ingredients have not been elucidated for most. The stanols and stanol esters, however, are available principally as purified extracts, approved for food use.

The regimen according to the second principal embodiment of the invention will entail administering the compositions of the invention in amounts and at intervals effective to provide a therapeutically significant reduction in CRP. The preferred regimens will be effective to reduce CVD risks and/or other conditions and diseases for which elevated CRP levels are an indicator.

In one form, the compositions of this alternative form of the invention will preferably contain 500 mg omega-3 rich refined fish oil, 100 mg resveratrol, 150 mg blueberry extract, 100 mg grape seed extract, 50 mg green tea extract, 100 mg gamma and delta tocopherol mixture (deodorize distillate). A dosage unit of this formulation will preferably be from 0.5 to 2.0 grams, preferably in gelatin or like capsules for oral administration. The capsules are desirably consumed in a regimen effective to lower levels of CRP, e.g., two to four times daily, at regular intervals, preferably taking them once in the morning and once in the evening or at intervals of eight hours. The doses and the individual ingredients can be varied by up to 50% of the above values, preferably varying by no more than 25%. The purity of the ingredients and the presence of added diluents, emulsifiers and other additives must be taken into consideration in determining the dosage.

In anther form, composition of the invention can comprise a mixture of green tea extract, e.g., from 30 to 60%, grape seed extract, e.g. from 20 to 40%, apple extract, e.g., from 10 to 20% and curcumin, e.g., from 2 to 10% as a dry powder. This form of mix can be mixed in the prepared meal, dip, or soup, or added before cooking/microwaving the meal or soup and is well delivered in foods comprising meat or meat substitute, especially with tomato, e.g., as Chili, taco and southern style meals and soups.

The above components of the compositions of the invention should have, individually, effective levels of purity to meet the objectives of the invention. For omega-3 fatty acids, this group includes linolenic, stearidonic, arachadonic, eicosapentaenoic (EPA), docosapentaenoic and docosahexaenoic (DHA) acids, but preferred mixtures of omega-3 fatty acids will include at least 50% of the fatty acids added by weight should as EPA or DHA. The other components, namely the resveratrol, blueberry extract, grape seed extract, green tea extract and gamma and delta tocopherol mixture, can be standardized for dosage level based on ORAC assay values (Oxygen Radical Absorbance Capacity assay (commonly referred to as the ORAC assay)), with the levels meeting those recommended for daily dosages. The above ingredients should be employed in total amount to provide a daily intake of at least 3,000 and preferably at least 5,000 ORAC units. Each of the components individually should contribute no less than 500 ORAC units. A therapeutic dosage unit will contain at least 1,000 and preferably at least 1,500 ORAC units. Preferably, each of the components individually contributes no less than 200 ORAC units.

In alternative dosage forms, the compositions can be mixed with suitable food ingredients to make a food or food mix. For example, they can be added to an acidulent, sweetener and flavor to provide a beverage mix for reconstitution with water, milk, juice or the like.

The following examples are presented to further explain and illustrate the invention and are not to be taken as limiting in any regard. Unless otherwise indicated, all parts and percentages are by weight.

### EXAMPLE 1

This example provides a preferred dosage form of a composition of the invention. Gelatin capsules are produced by preparing a mixture of the following ingredients by grinding under a vacuum to assure intimate mixing and a dry character, and then filling individual gelatin capsules with a total of 1000 mg as follows:

50 mg curcumin

60 mg bilberry extract

250 mg grape seed extract

375 mg green tea extract

125 mg apple extract

These capsules are consumed in a regimen effective to lower levels of one or more key indicators, preferably taking once in the morning and once in the evening.

### EXAMPLE 2

This example provides a preferred dosage form of an alternative composition of the invention. Gelatin capsules are produced by the process and formulation of Example 1, but this time 1000 mg of plant-derived sterols are added. The regimen remains the same.

### EXAMPLE 3

This example illustrates a method employed to determine the effect of anti-inflammatory properties of various plant derived extracts by measuring the inhibition of PGE2 biosynthesis by test materials. The tests were performed using cultured Human Coronary Artery Smooth Muscle Cells. The new sample preparation and LC-MS/MS method were developed for quantification of PGE2 using stable isotope dilution. Briefly, the method involves spiking the cell media with internal standard (PGE2-d4), mixing the samples (100 μl) with acetonitrile (400 μl), removing precipitated proteins by filtration using 96 filtering plate (0.45 μm), and concentration filtrates to ˜50-75 μl in Speed vac centifuge. After addition of 25 μl of mobile phase A (15% MeCN+0.25% Et₃N, v/v) the samples were injected on a pre-equilibrated BetasilBasic C18 3 μm guard column (2.1×10 mm) connected to a two way switching valve. The hydrophilic constituents presented in the cell culture media were washed to the waste by pumping mobile phase A (20 sec), after which the guard column was connected to a BetasilBasic C18 separation column (2.1×30 mm). The prostaglandins were eluted using mobile phase B (70% MeCN+0.25% Et₃N). The PGE were detected using LTQ ion trap equipped with electrospray interface operating in negative mode. Prostaglandins were quantified in Single Reaction Monitoring mode. The cell viability, at the end of the experiment, was determined using a CellTeck kit according to the manufacturer instructions.

### EXAMPLE 4

This example provides a preferred dosage form of a composition of the invention. Gelatin capsules are produced by preparing a mixture of the following ingredients by grinding under a vacuum to assure intimate mixing and a dry character, and then filling individual gelatin capsules with a total of 1000 mg as follows:

500 mg omega-3 rich refined fish oil

100 mg resveratrol

150 mg blueberry extract

100 mg grape seed extract

50 mg green tea extract

100 mg gamma and delta tocopherol mixture (deodorize distillate)

These capsules are consumed in a regimen effective to lower levels of CRP, preferably taking once in the morning and once in the evening.

### EXAMPLE 5

This example provides a preferred dosage form of an alternative composition of the invention. Gelatin capsules are produced by the process and formulation of Example 1, but this time 1000 mg of plant-derived sterols are added. The regimen remains the same.

### EXAMPLE 6

This example provides a preferred dosage form of an alternative composition of the invention. Here, the following formulation is used to prepare candy products by preparing a candy melt at 332° F., mixing in the actives, pouring the candy melt onto a candy table mixing in the flavor, and then shaping and cooling. Following cooling the candies are tested by HPLC for the presence of actives with the results as reported below.

| Ingredient | Amount |
|---|---|
| Maltitol Syrup-Lycasin, | 0.40219 |
| 75% solution | |
| Isomalt type PF | 2.81537 |
| Water | 0.03341 |
| Pectin | 0.00835 |
| Flavor, cherry | 0.0142 |
| Water | 0.6933 |
| Citric acid | 0.02047 |
| Actives | 0.01222 |
| Apple extract¹ | |
| Bilberry extract | |
| Curcumin powder² | 0.00066 |
| Grape seed extract | |
| Green tea extract³ | |

¹Apple Extract contains 13.07% chlorogenic acid by HPLC
²Curcumin Powder contains 91.9% curcuminoides by HPLC
³Green Tea Extract contains 3.78% caffeine by HPLC

Following cooling, the candies are tested by HPLC for the presence of actives with the results as reported below.

HPLC Testing Results of Hard Candy with Actives

| | Amount | | |
|---|---|---|---|
| | (mg/4 g Hard Candy) | | |
| Ingredient | Cherry | Peppermint | Cherry |
| Curcuminoides | 1.07 | 1.03 | 0.61 |
| Curcumin Powder³ | 1.15 | 1.11 | 0.66 |
| Chlorogenic Acid | 0.47 | 0.45 | 0.26 |
| Phloridzin | 2.48 | 3.03 | 1.05 |
| Apple Extract¹ | 3.6 | 3.44 | 1.99 |
| Caffeine | 0.35 | 0.35 | 0.19 |
| EGCG | 3.98 | 5.55 | 2.5 |
| Green Tea Extract² | 9.05 | 9.05 | 5.03 |

Note:
same footnotes as above

### EXAMPLE 7

This example repeats the procedure of Example 6, but this time the ingredients include 0.1 parts of mixed tocopherols.

### EXAMPLE 8

This example illustrates the application of one formulation of the invention (Mix-1, below) in Boca® Meatless Chili manufactured by Kraft Foods. The meal was prepared by microwave heating according to the directions on the package. 300 mg Mix-1 powder (mixture of 48.7% green tea extract, 30.7% grape seed extract, 14.6% apple extract, and 6.0% curcumin), which is equal to a quarter of daily dosage, was mixed into a half serving of the Meatless Chili. The Meatless Chili with Mix-1 was found with no difference in taste and flavor compared to the control meal. A slightly yellowish color, due to curcumin, was almost indistinguishable in the meatless chili with Mix-1.

### EXAMPLE 9

This example illustrates the application of one formulation of the invention (Mix-1) in Southwest Bean Soup manufactured by Tabatchnick. Two soup bags were heated in boiling water according to the directions. 600 mg of Mix-1 was then mixed into one soup bag. The soup with Mix-1 was found very tasty having no difference in taste to the soup without adding Mix-1. The color of the soup with Mix-1 slightly turned to yellowish.

### EXAMPLE 10

This example illustrates the application of one formulation of the invention (Mix-1) in Banquet® Macaroni & Beef Meal manufactured by Conagra Foods. One serving of the meal was heated in microwave according to the directions. After mixing the meal and sauces, the meal was equally divided into two portions. 300 mg Mix-1 was mixed into one portion of the meal. The two portions were then tasted and found essentially indistinguishable.

### EXAMPLE 11

This example illustrates the application of the Mix-1 of the invention in Weight Watcher Lasagna Bolognese manufactured by Heinz. One serving of the Lasagna was prepared by microwave according to the directions. After mixing with sauces, the meal was equally divided into two portions. To one portion 300 mg Mix-1 was mixed into one portion. The portion with Mix 1 was found to have no objectionable taste. Color slightly turned yellowish.

### EXAMPLE 12

This example illustrates the application of the Mix-1 of the invention in Taco meat. 305 mg Mix 1 was mixed into a half serving of prepared warm Taco meat (130 g). Compared to the portion without Mix-1, the one with the Mix-1 added was found to be no different in taste and flavor but showed a slight change in color (yellowish).

### EXAMPLE 13

This example illustrates the application of the Mix-1 of the invention in Tostitos® Hot Salsa. 300 mg Mix-1 was mixed into 2 tsp (33 g) Salsa. It was noted that the Salsa with added Mix-1 was changed in color and flavor, but not objectionably. A slightly dry sensation on the tongue was observed as compared to the control.

### EXAMPLE 14

This example illustrates the application of the Mix-1 of the invention in Cedar's Lovers Hommus. 150 mg Mix-1 was mixed into 2 tsp (31 g) Hommus. Compared to the original product, there was no significant difference in taste and flavor. Color was off slightly but still observed as almost indistiguishable.

### EXAMPLE 15

This example illustrates the application of Mix-1 of the invention in Creamy French Dressing (Kraft). 150 mg and 300 mg of Mix-1 were mixed into two test samples of 2 tsp (31 g) of dressing. Compared to control, either test sample gave a sharp hit and dry sensation on the tongue, but this taste quickly disappeared. Color changed to more reddish from yellowish. Texture thickens up.

### EXAMPLE 16

This example illustrates the application of Mix-1 of the invention in Lipton Cup-a-Soup®, Spring Vegetable Instant Soup. Mix 600 mg Mix 1 with one bag (one serving) dry powder, following the directions by adding 6 fl oz boiling water and stirring until noodles were tender. The soup with Mix-1 had a good appearance, and was slightly changed the flavor, in comparison to the control soup.

### EXAMPLE 17

This example illustrates the application of Mix-1 of the invention in Ensure® high protein shake (wild berry flavor). Mix and disperse 600 mg Mix1 into one serving shake. It was found that the shake with Mix-1 was similar in taste and flavor as the shake without Mix-1.

### EXAMPLE 18

This example illustrates the application of Mix-1 of the invention to Hot Cocoa Mix (Nestle's Rich Chocolate Flavor). 600 mg Mix-1 was mixed and dispersed with one serving powder, and then 6 ounces of hot water was added and stirred. The drink with Mix-1 masks the cocoa flavor, adds a slight tea flavor, and changes the color slightly yellower when compared to a control drink.

### EXAMPLE 19

This example illustrates the application of the Mix-1 of the invention to Granola having the following Formula:

| Ingredient | Amount (g) |
|---|---|
| Quick oats | 140 |
| Crisp rice | 60 |
| Non fat dry milk | 4 |
| Cinnamon | 5 |
| Mix fruits | 27.5 |
| Almond flakes | 20 |
| Salt | 1.25 |
| Oil | 45 |
| Liquid sugar(67.5) | 135 |
| Molasses | 5 |
| Honey | 12.5 |
| Mix-1 | 7.25 |

The granola was prepared by properly mixing all ingredients and baking. The granola was tasted and well accepted.

### EXAMPLE 20

This example illustrates the application of the Mix-2 of the invention in Ensure® g high protein shake (wild berry flavor). Mix and disperse 600 mg Mix-2 (mixture of 38.0% green tea extract, 24.0% grape seed extract, 21.9% bilberry extract, 11.4% apple extract, and 4.7% curcumin) into one serving shake. The shake with Mix-2 had a good berry flavor and actually tasted better than the shake without adding Mix-2; however, the color changed from pink to purple.

### EXAMPLE 21

This example illustrates the application of the Mix-2 of the invention in Ghirardelli® Chocolate Syrup Brownies. One box of Ghirardelli Brownies Mix was mixed with egg and vegetable oil according to the direction; the batter was prepared and divided into two equal portions; to one portion 300 mg Mix-2 was added. The two different portions were baked according to directions. The two prepared Brownies were tasted. The Brownie with Mix 2 was dark in color and was not as sweet as control. There was no objectionable flavor or taste.

### EXAMPLE 22

This example illustrates the application of the Mix-1 of the invention in yogurt prepared according to the formula below. The milk was heated to 180° F., allowed to cool to 115° F. and blended with the remainder of the ingredients including the Mix-1. The mixture was then separated equally into 8 yogurt culturing cups and cultured in accordance with the yogurt manufacturer's directions for 9 hours. The yogurt was then refrigerated. The Mix-1 yogurt had no objectionable taste and the color was very pleasant.

| Ingredient | Amount | |
|---|---|---|
| Milk, 2% fat | 1000 | ml |
| Maple syrup | 25 | ml |
| Honey | 25 | ml |
| Non-fat dry milk | 25 | g |
| Sucrose | 30 | g |
| Mixed fresh berries | 200 | g |
| Commercial yogurt starter | 10 | g |
| Mix 1 or Mix 2 | 600 | mg |

### EXAMPLE 23

This example illustrates the application of the Mix-2 of the invention in yogurt prepared according to the formula above. The procedure of Example 22 was repeated, this time using Mix-2 in place of Mix-1. The Mix-2 yogurt had no objectionable taste and the color was very pleasant.

The above description is intended to enable the person skilled in the art to practice the invention. It is not intended to detail all of the possible modifications and variations which will become apparent to the skilled worker upon reading the description. It is intended, however, that all such modifications and variations be included within the scope of the invention which is seen in the above description and otherwise defined by the following claims. The claims are meant to cover the indicated elements and steps in any arrangement or sequence which is effective to meet the objectives intended for the invention, unless the context specifically indicates the contrary.

1. A therapeutic composition comprising a blend of food extracts that delivers catechins, proanthocyanidins and curcumin which are effective for the purpose of reducing markers of chronic inflammation.

2. A therapeutic composition according to claim 1, wherein the food extracts are selected from the group of apple extract, green tea extract, curcumin, bilberry extract, blueberry extract, mixed tocopherols, resveratrol, omega-3 rich oils and grape seed extract, containing compositions with anti-inflammatory activity, the compositions being present in amounts individually and combined to provide a therapeutically significant reduction of at least two of the markers selected from the group of CRP, COX-2, 5-LOX, TNF-α, NF-κB, IL-6 and IL1-β.

3. A therapeutic composition according to claim 1, which comprises curcumin, bilberry extract, grape seed extract, green tea extract and apple extract.

4. A therapeutic composition according to claim 3, which comprises from about 30 to 60% green tea extract, from about 20 to 40% grape seed extract, from about 10 to 20% apple extract, and from about 2 to 10% curcumin.

5. A therapeutic composition according to claim 3, which comprises a dry powder.

6. A therapeutic composition according to claim 1, which comprises omega-3 rich refined fish oil, resveratrol, blueberry extract, grape seed extract, green tea extract, gamma and delta tocopherol mixture.

7. A therapeutic composition according to claim 1, which comprises ingredients that deliver chlorogenic acid for the reduction of chronic inflammation.

8. A therapeutic composition according to claim 1, which comprises a source of anthocyanins for the reduction of chronic inflammation.

9. A therapeutic composition according to claim 1, which reduces the expression and/or activity of enzymes in the COX-2 pathway.

10. A therapeutic composition according to claim 1, which reduces the expression and/or activity of enzymes in the COX-2 pathway thereby reducing the presence of prostaglandin end products as measured in the serum and urine.

11. A therapeutic composition according to claim 1, which reduces 5-LOX activity in vivo.

12. A therapeutic composition according to claim 1, which reduces the expression and/or activity of enzymes in the 5-LOX pathway thereby reducing the presence of prostaglandin end products as measured in the serum and urine.

13. A therapeutic composition according to claim 1, which reduces the production of IL-6.

14. A therapeutic composition according to claim 1, which reduces the expression IL1β.

15. A therapeutic composition according to claim 1, which reduces the expression interleukins TNF-α and IFN-γ and related inflammatory pathways.

16. A therapeutic composition according to claim 1, which reduces the expression of NFκB and related inflammatory pathways.

17. A therapeutic composition according to claim 1, which reduces the expression of iNOS in endothelial tissue and related inflammatory pathways

18. A therapeutic composition according to claim 1, which reduces the expression of COX-2 and/or PGE-2 production in cardiac smooth muscle tissue.

19. A therapeutic composition according to claim 1, which reduces oxidative markers in the plasma or serum as measured by TBARS.

20. A therapeutic composition according to claim 1, which reduces the presence of reactive oxygen species in the blood.

21. A therapeutic composition according to claim 1, which reduces the oxidation of lipoproteins.

22. A therapeutic composition according to claim 1, which reduces the expression of selectins ICAM-1 and VCAM-1.

23. A therapeutic composition according to claim 1, which reduces the expression of the COX-2 inflammation pathway and reduces the reactive oxygen species in the blood and urine.

24. A therapeutic composition according to claim 1, which reduces the expression of COX-2 inflammation pathway and iNOS production in the entholium.

25. A therapeutic composition according to claim 1, which reduces the expression of COX-2 inflammation pathway and the production of leukotrines by the 5-LOX pathway.

26. A therapeutic composition according to claim 1, which reduces the expression of NFκB inflammation pathway and reduces reactive oxygen species in the serum.

27. A therapeutic composition according to claim 1, which reduces the expression of TNF-α inflammation pathway and the production of iNOS in the endothelium.

28. A therapeutic composition according to claim 1, which reduces the cascade of inflammatory responses resulting from increased levels of IL-6.

29. A therapeutic composition according to claim 1, which reduces the expression of inflammatory markers produced by adipose tissue associated with being overweight or obese including IL-β, IL-6, IL-8 and IL-10.

30. A therapeutic composition according to claim 1, which reduces the expression of inflammatory markers produced by adipose tissue associated with being overweight or obese.

31. A therapeutic composition according to claim 1, which reduces the expression of inflammatory markers produced by adipose tissue associated with being overweight or obese which result in inflammatory cardiovascular disease associated with diabetes.

32. A therapeutic composition according to claim 1, which reduces the expression of inflammation associated with gastric reflux disease.

33. A therapeutic composition according to claim 1, which reduces the expression of inflammation associated inflammatory bowel diseases.

34. A therapeutic composition according to claim 1, which reduces the expression of inflammation associated colonic polyposis and colon cancer.

35. A therapeutic composition according to claim 1, which reduces the expression of inflammation associated joint discomfort from rheumatoid arthritis.

36. A therapeutic composition according to claim 1, which reduces the expression of inflammation associated osteoarthritis.

37. A therapeutic composition according to claim 1, which reduces the effects of inflammation associated osteoarthritis when combined with glucosamine.

38. A therapeutic composition according to claim 1, which reduces the effects of inflammation associated osteoarthritis when combined with glucosamine and chondroitin sulfate.

39. A therapeutic composition according to claim 1, which further contains: plant sterols and/or plant sterol esters in amounts effective to lower serum cholesterol.

40. A therapeutic dosage unit which contains from 0.1 to 5.0 grams, preferably 1 to 3 grams, of the composition of claim 1 in gelatin or like capsules for oral administration.

41. A therapeutic dosage unit according to claim 40, which contains sufficient active ingredients to provide at least 1,000 and preferably at least 1,500 ORAC units.

42. A therapeutic dosage unit according to claim 41, wherein each of the components individually contributes no less than 200 ORAC units.

43. A therapeutic regimen comprising administering the compositions of claim 1 in amounts and at intervals effective to provide a therapeutically significant reduction in C-reactive protein.

44. A therapeutic regimen comprising administering a composition according to claim 41 at intervals and in amounts effective to reduce cardiovascular disease risks and/or other conditions and diseases for which elevated CRP levels are an indicator.

45. A therapeutic composition as in claim 1 in combination with 0.5 to 5 g of glucosamine to reduce joint inflammation, reduce pain from osteoarthritis and enhance joint health.

46. A therapeutic composition as in claim 1 with 0.1 to 1 g of chondroitin sulfate.

47. A food product containing the therapeutic composition according to claim 1.

48. A food product according to claim 47, which comprises yogurt.

49. A food product according to claim 47, which comprises meat and tomato sauce.

50. A food product according to claim 47, which comprises soup.
